(19) United States
(12) Patent Application Publication
(10) Pub. No.: US 2006/0121461 A1
(43) Pub. Date: Jun. 8, 2006

(54) Methods for identifying and isolating unique nucleic acid sequences

(76) Inventors: Carsten Harms, Bremerhaven (DE); Holger Maul, Hannover (DE); William W. Au, Webster, TX (US); Boris Oberheitmann, Bremen (DE)

Correspondence Address: Peter Rogalskyj;Rogalskyj & Weyand, P O Box 44, Livonia, NY, 14487-0044, US

(21) Appl. No.: 10/512,831

(22) PCT Filed: Apr. 30, 2003
(86) PCT No.: PCT/EP03/04570

§ 371 Date: Aug. 5, 2005

Related U.S. Application Data
(60) Provisional application No. 60/376,630, filed on Apr. 30, 2002.

(51) Int. Cl. C12Q 1/68; C12P 19/34

(52) U.S. Cl. 435/6; 435/91.2

(57) ABSTRACT

A subtractive suppression hybridization (SSH) assay and uses thereof are described. In particular, methods of identifying and isolating nucleic acid sequences, which are unique for a certain cell, tissue or organism are provided, wherein said unique nucleid acid sequences are related to for example diseases genes. More specifically, SSH assays for unique genomic DNA sequences and improved SSH assays that are combined with 2D gel electrophoresis techniques are provided. The presented methods are particular useful for the identification of genes involved in the development of various diseases, including cancer, hypertension and diabetes as well as for monitoring animals and food, for example for infection agents and other contaminants.

### FIELD OF THE INVENTION

The present invention relates to a subtractive suppression hybridization (SSH) assay and uses thereof. In particular, the present invention relates to methods of identifying and isolating nucleic acid sequences, which are unique for a certain cell, tissue or organism, wherein said unique nucleid acid sequences are preferably related to genes that are etiologically related to a disease. More specifically, the present invention is directed to SSH assays for unique genomic DNA sequences and to improved SSH assays that are combined with 2D gel electrophoresis techniques. The presented methods are particular useful for the identification of novel genes involved in the development of various diseases, including cancer, hypertension and diabetes as well as for monitoring animals and food, for example for infection agents and other contaminants.

### BACKGROUND ART

Currently, there are two basic approaches to determine the relationship between abnormal genes and disease. On the one hand, molecular procedures such as DNA sequencing are used to identify mutations in genes that have already been suspected to be associated with certain disease. The availability of these genes for mutational analyses is, however, very limited. On the other hand, DNA arrays enabled researchers to survey the genome for differences in expression of genes. As a result, it is common to identify up to 20,000 candidate genes. Due to the large number of candidate genes, experimental variations and availability of appropriate software become the limiting factors in associating, certain expression patterns with disease. Several methods have been used to distinguish differences between two organisms or two genomes, but have not been used in identification of disease genes. The first method is called DSC (Differential Subtraction Chain). This most recent method deploys several hybridization steps to compare two nucleic acid populations (driver and tester) after they were enriched by PCR (Luo et al. 1999). Understandably, significant changes in the nucleic acid populations used for the comparison are initiated by the PCR at the very beginning of the method. One cannot assure the exact amplification of the tester and driver DNA used for further comparison. Therefore, differences introduced by the initiated PCR will consequently lead to artificial differences after hybridization. The second method is called RDA (representational difference analysis) and it employs a representational sampling, approach by cutting the DNA into fragments based on its restriction enzyme cutting pattern, and attaching these restriction fragments to a PCR adapter for PCR amplification. This method is also dependent upon selective enrichment of differences between organisms but does not use any physical separation. This method is found to be very time consuming and not suitable for routine analysis (Lisitsyn et al. 1993; Hubank, et al. 1999).

It would thus be desirable to provide means and methods which are capable of identifying genes that are absent in healthy but present in disease tissues directly, without prior knowledge on the function of the genes. The solution to said technical problem is achieved by providing the embodiments characterized in the claims, and described further below.

### DESCRIPTION OF THE INVENTION

In one aspect, the present invention generally relates to a subtractive suppression hybridization (SSH) assay for unique genomic DNA sequences and uses thereof. In particular, the present invention relates to a method of identifying and/or isolating a nucleic acid fragment or a corresponding gene which is unique for a certain cell, tissue or organism, comprising the steps of:
(a) dividing a tester nucleic acid sample into a first and a second nucleic acid sample, wherein said nucleic acids comprise or substantially consist of eukaryotic genomic DNA fragments;
(b) attaching a first PCR suppression adapter to each end of a DNA fragment in said first nucleic acid sample and attaching a second PCR suppression adapter to each end of a DNA fragment in said second nucleic acid sample;
(c) contacting each of said first and second nucleic acid samples separately with a driver nucleic acid sample;
(d) denaturing and reannealing said DNA fragments;
(e) combining said first and second nucleic acid samples to form a mixture of nucleic acids;
(f) contacting said nucleic acid mixture with a first nucleic acid primer comprising a nucleotide sequence that is complementary to a nucleotide sequence of said first adapter and contacting said nucleic acid mixture with said second nucleic acid comprising a nucleotide sequence that is complementary to a nucleotide sequence of said second adapter;
(g) adding to said mixture obtained after step (f) an effective amount of reagents necessary for performing a PCR; and
(f) cycling the mixture obtained after step (g) through at least one cycle of the denaturing, annealing and primer extension steps of PCR, wherein amplification of non-unique nucleic acid fragments is suppressed during PCR.

The method of the present invention is based on experiments relating to the characterization of integrated DNA fragments of unknown base sequences of foreign origin using the genetically modified mold, Penicillium nalviogense. In particular, it could be surprisingly shown that detection and characterization of foreign genes in a eukaryotic genomic background can be achieved by suppression subtractive hybridisation method (SSH); see Examples 1 and 2. Allmost each DNA fragment derived from the tester genomic DNA was found to be completely absent in the driver organism.

The invention includes the rapid enrichment of differences between two organisms and is a substantial improvement of a technique published by Diatchenko, et al. (1999). Suppression subtractive hybridisation (SSH) is a cost-effective and powerful technique for the isolation of species-specific DNA sequences from closely related microorganisms. The principle behind this technique is a two-step hybridization with an excess of genomic DNA from a “driver” organism compared with that from a “tester” organism. After reannealing tester and driver DNA, only specific DNA fragments (tester DNA) with an appropriate pairs of adapter can participate in an exponential PCR amplification when defined oligonucleotide is used as primer. Next, a secondary PCR amplification is performed using nested primers to further reduce background PCR products and enrich for tester-specific sequences.

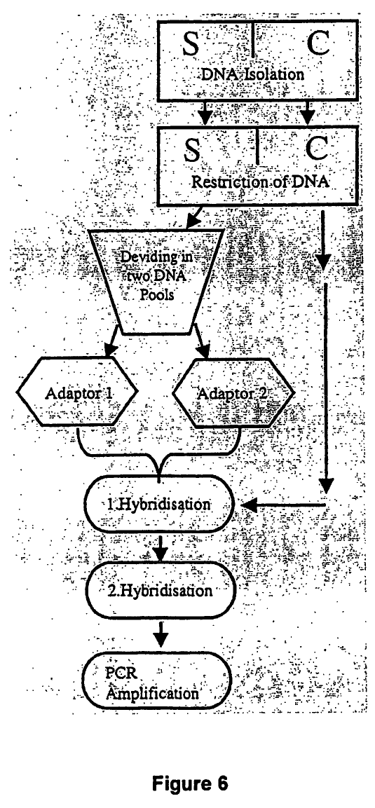

An improvement of this invention is the application of the subtractive suppression hybridization to human genomic DNA although this method was designed originally to enrich unique sequences between microorganisms obtained by conversion of mRNA to cDNA. The technique has never been applied to higher eukaryotes because of the high complexity of the genomic DNA. So far, for investigations of differences between closely related organisms, all approaches have been based on RNA. The basic procedural steps of the method present invention are shown in FIG. 6.

Hence, in contrast to for example the cDNA SSH assays described in the prior art, the method of the present invention is not primarily directed to analysis of expression pattern of for example differentially induced cells, a cell such as a tumor cell that acquired uncontrolled cell proliferation may also be regarded as being in an induced state, but to the analysis of particular phenotypes due to a different genomic background, including the detection of for example foreign genetic material such as (intergrated) viral DNA or parasites.

Adapters that can be used in accordance with the present invention are described, for example, in Diatchenko, et al. (1996; 1999) and WO96/23079, the disclosure contents of which are incorporated herein by reference. The adapters can be composed of either DNA or RNA and can be either single-stranded or double-stranded when attached to the DNA fragment. In a preferred embodiment, the adapters are at least partially double-stranded to aid in ligation of the adapter to the DNA fragment. The adapters can be attached to the ends of DNA or RNA fragments using a variety of techniques that are well known in the art, including DNA ligase-mediated ligation of the adapters to sticky- or blunt-ended DNA, T4 RNA ligase-mediated ligation of a single-stranded adapter to single-stranded RNA or DNA, oligo (dA) tailing using terminal transferase, or via any DNA polymerase (or a reverse transcriptase if RNA is the template) using a primer having a sequence which corresponds to the adapter sequence. As used herein, the term “attach,” when used in the context of attaching the adapter to a DNA fragment, refers to bringing the adapter into covalent association with the DNA fragment regardless of the manner or method by which the association is achieved. Using the teachings contained herein and in the prior art, the person skilled in the art could readily construct other adapters that have different sequences from those adapters exemplified herein, including variants of the subject adapters, that would be operable with the subject invention. Any polynucleotide sequence that comprises a primer binding portion and an effective suppressor sequence portion and which when associated with a DNA or RNA fragment can form a suppressive “pan-like” structure during PCR as described in Diatchenko, et al. (1996; 1999) and WO96/23079 is contemplated by the subject invention, such the Type 1 and Type 2 adapter structures described therein.

Preferably, the adapter should not contain any sequences that can result in the formation of “hairpins” or other secondary structures in the DNA which can prevent adapter ligation or primer extension. As would be readily apparent to a person skilled in the art, the primer binding sequence portion of the adapter can be complementary with a PCR primer capable of priming for PCR amplification of a target DNA.

Preferably, the primers of the subject invention have exact complementarity with the adapter sequence. However, primers used in the subject invention can have less than exact complementarity with the primer binding sequence of the adapter as long as the primer can hybridize sufficiently with the adapter sequence so as to be extendable by a DNA polymerase. As used herein, the term “primer” has the conventional meaning associated with it in standard PCR procedures, i.e., an oligonucleotide that can hybridize to a polynucleotide template and act as a point of initiation for the synthesis of a primer extension product that is complementary to the template strand.

Design of adapters and primers as well as the choice of appropriate hybridization conditions can be performed according to known methods, see, e.g., Nucleic Acid Hybridization (1985) Ed. James, B. D. & Higins, S. J. (IRL Press Ltd., Oxford); Lukyanov et al. Bioorganic Chem. (Russian) 20 (1994), 701-704; Siebert et al. Nucleic Acids Res. 23 (1995), 1087-1088; Clontech PCR-Select cDNA Subtraction Kit 7 rxns K1804-1; PCR-Select Differential Screening Kit each K1808-1; Custom Clontech PCR-Select Subtraction: Level I each CS1103; Custom Clontech PCR-Select Subtraction Differential Screening: Level II each CS1104; Custom Clontech PCR-Select SMART Amplification CS1105 (Clontech 1020 East Meadow Circle Palo Alto, Calif. 94303-4230 USA) and the appended examples.

The adapters and primers used in the subject invention can be readily prepared by the person skilled in the art using a variety of techniques and procedures. For example, adapters and primers can be synthesized using a DNA or RNA synthesizer. In addition, adapters and primers may be obtained from a biological source, such as through a restriction enzyme digestion of isolated DNA. The primers can be either single- or double-stranded. Preferably, the primers are single stranded. In a particular preferred embodiment of the methods of the present invention, said adapters or nucleic acid primers comprise a nucleotide sequence comprising a restriction endonuclease recognition site.

In an independent aspect of the present invention described further below, the SSH method was combined with a special two dimensional polyacrylamide gel-electrophoretic (PAGE) technique by which the usual background was eliminated from the prospective foreign PCR fragments according to their base composition (Müller et al., Nucl. Acids Res. 9 (1981), 95-118); see Examples 1 and 2. Hence, this improved method finds its use also for the detection and characterization of unique nucleotide sequences among cDNA, prokaryotic DNA and viral DNA or fragments thereof. DNA fragments obtained by the methods of the invention were suitable for direct DNA sequencing.

The methods of the present invention have been verified, for example, by using eukaryotic DNA (Aspergillus niger DNA interblended with Lambda DNA), where only interblended Lambda DNA fragments were isolated. Furthermore, efficiency of the technique was demonstrated by the purity and specificity of the fragments, which were suitable for sequencing after the second electrophoretic separation, without clean-up procedure; see also Example 2.

The methods of the present invention can primarily be used to compare genomic sequences, preferably of eukaryotic origin, from different cells/tissues/organisms with the purpose of identifying unique gene sequences. Furthermore, the improved methods of the present invention described herein can be used to identify differences in gene expression from cells/tissues/organisms using, RNA and/or cDNA. In addition, the methods of the present invention can be used to identify minute differences between similar organisms such as infectious agents to obtain sequence data of antibiotic resistance Genes for improving the efficaciousness of treatments. In an important aspect of the instant invention the disclosed methods can be used to identify differences in two different genomes which are very closely related to obtain genes involved in the development of various diseases, including cancer, hypertension, and diabetes as well as differences in gene expression that are relevant to disease and/or caused by exposure to toxicants.

Disease causing genes, for example, that are identified by a method of the present invention can be used as sentinel biomarkers to indicate exposure to toxicants and to assess risk for health problems. Similarly, said identified disease causing genes can be used for disease prevention.

As described in the examples, the methods of the present invention are preferably perfomed, wherein said first and second nucleic acid samples are each separately contacted with an excess of a third nucleic acid sample, i.e. driver DNA after performing step (b) but prior to performing step (c). Usually, said driver nucleic acid sample comprises nucleic acid sequences that are complementary with at least one nucleic acid fragment in said first and second nucleic acid samples.

After attching the adapters and after denaturing and reannealing of said nucleic acid fragments step (c) preferably further comprises filling in any single-stranded portions of said adapter, wherein said adapter and said nucleic acid fragment comprise nucleic acid that is double-stranded. As described in the examples, said nucleic acid fragments may be less than 500 bp in length. However, different lengths of said nucleic acid fragments may be used as well. The DNA fragments used in the subject invention can be obtained from DNA by random shearing of the DNA, by digestion of DNA with restriction endonucleases, or by amplification of DNA fractions from DNA using arbitrary or sequence-specific PCR primers. In one embodiment of this method, genomic DNA is fragmented, preferably using restriction enzymes. Preferably, the restriction endonuclease is RSAI. However, other restriction endonucleases may be used as well, preferably 4- to 6-cutters.

In one embodiment, the methods of the present invention are performed such that said nucleic acids of said tester or driver nucleic acid sample are immobilized or suspended on a chip or microarray. Chip and array technology are well known to the person skilled in the art. Advances in approaches to DNA-based diagnostics are reviewed, for example, by Whitcombe et al. in Curr. Opin. Biotechnol. 9 (1998), 602-608. Furthermore, DNA chips and microarray technology devices, systems, and applications are described by, e.g. Cuzin, Transfus. Clin. Biol. 8 (2001), 291-296 and Heller, Annu. Rev. Biomed. Eng. (2002), 129-153. Furthermore, active microelectronic array systems for DNA hybridization, genotyping and pharmacogenomic applications (see, e.g., Sosnowski, Psychiatr. Genet. 12 (2002), 181-192) and chips and detection methods on the basis of DNA conformational switches as sensitive electronic sensors of analytes can be employed in accordance with the present invention; see, e.g., Fahlman and Sen, J. Am. Chem. Soc. 124 (2002), 4610-4616.

In a preferred embodiment of the present invention, the described method is perfomed with a driver nucleic acid sample comprising a pool of nucleic acids. This measure is particularly useful for pin-pointing a gene which is most likely responsible for a certain phenotype. For example, in order to identify a disease causing gene a tester nucleic acid sample obtained from a patient is screened against a driver nucleic acid sample comprising nucleic acids from several healthy subjects of different cultural background in order exclude the amplification of nucleic acid sequences that are unique because of lineage and descent. Hence, it is also an object of the present invention to provide such driver samples of pooled nucleic acids, for example in a kit useful for performing the method of the present invention and generally applicable for approaching the genotype for any particular phenotype. In this novel aspect of SSH assays, the methods described herein are directed generally to the identification and isolation of unique target sequences, wherein said nucleic acids of said tester nucleic acid sample comprise or substantially consist of fragments of prokaryotic or viral DNA.

As mentioned before, the cells, tissue or organisms investigated may display different phenotypes such as a symptom of a disease. However, it is to be understood that the methods of the present invention are also particularly useful for the identification and isolation of “hidden” nucleic acids, which do not or at least not at the onset of their presence display an observable phenotype, for example in genetic predispositions, contamination of foods, and infected animals.

The methods of the present invention are particularly powerful when samples are used, which are derived from the same or similar species, in partiuclar if said samples are derived from the same or related subjects, for example twins.

Since the method of the present invention has been proven to be particularly useful for the analysis of complex genomes, the samples are preferably derived from a vertebrate or a plant. In the latter embodiment, the methods of the present invention are especially useful in plant breeding, for example in identifying pathogen resistance genes. In the preceding embodiment, said vertebrate is preferably a mammal or a fish; particularly human is preferred; see also Example 2.

In one important aspect of the present invention, the subtractive suppression hybridization assay (SSH) described herein is to identify genes that are etiologically related to a disease. With this technique, DNA samples from disease specimens will be hybridized with samples from normal specimens to identify DNA sequences that are present or absent the disease specimens. These sequences will be analyzed further to elucidate their functions that may be causally related to the disease. Accordingly, in this embodiment said tester nucleic acid sample is derived from diseased tissue and said driver nucleic acid sample is derived from healthy tissue or vice versa. Hence, it is expected that said unique nucleic acid fragment identified by a method of the present invention corresponds to a disease causing gene.

In a preferred embodiment of the present invention, said unique nucleic acid fragment or corresponing gene identified or isolated is present in the diseased tissue and absent in the healthy tissue or vice versa. Furthermore, information generated from the SSH will be used to design DNA arrays and/or chips which will be used to monitor populations for (1) clinical role of the genes for the same disease in different regions around the world, (2) early diagnosis of disease, (3) response to therapy, and (4) assessment of health risk.

However, as mentioned before, the methods of the present invention are not restricted to analysis of disease related phenotypes but encompass the analysis of any genotypic difference between at least two subjects. Those subjects may differ also in their phenotype which may be any phenotype that can be recognised or measured in any way, but preferably observable by the eye. Those phenotypes typically include economically important phenotypes, i.e. traits, in particular if those traits are multigenetically inherited. This makes the method of the present invention particularly useful in plant and animal breeding.

As mentioned before, the present invention in an independent aspect relates to a method of applying the general SSH assay in combination with a further step of subjecting the PCR fragments to 2D gel electrophoresis.

As described in the examples, a new combined technique is provided comprising SSH and a specific two dimensional polyacrylamide gel electrophoresis that reduces the unspecific PCR fragment background even when lower eukaryotes such as the mold P. nalgiovense are analysed. Because the specific tester DNA fragments were demarcated from the background it was possible to directly identify the fragments by sequencing.

The principle of the two-dimensional gel electrophoresis technique is based on a separation of DNA fragments according to their base composition and fragment size. After one-dimensional PAGE the PCR fragments were excised and positioned perpendicularly on a polyacrylamide gel to run in the second dimension, using a buffer that contains a high molecular weight dye (bisbenzimide-PEG or PEGIII). This benzimide dye intercalates specifically with AT clusters (PEGI) or with GC clusters (PEGIII) and retards the electrophoretic migration of DNA fragments in proportion to their relative AT (GC) content (Mueller et al. (1981) and Harms et al. (2000)). Consequently, AT-cluster (GCcluster) rich fragments produce spots in the gel situated above (or underneath when using PEG III) those spots from DNA fragments that contain AT/GC ratios near 1.

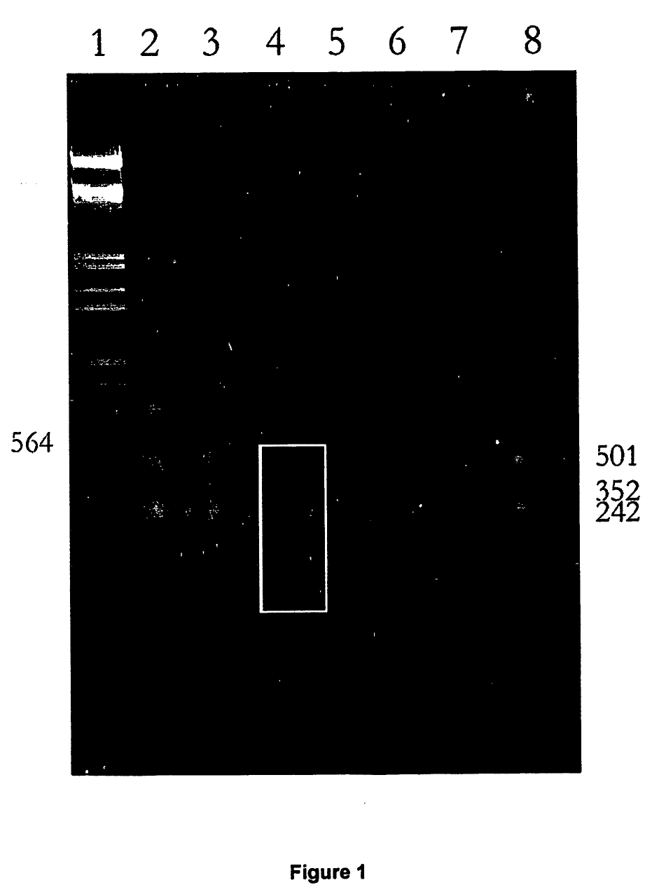

In Example 1, due to the short DNA fragments (<500bp) used for the SSH method it was decided to separate the PCR fragments on a two-dimensional polyacrylamide gel containing a bisbenzimide dye. As shown in FIG. 1 SSH-fragments could also be separated on agarose gel, but when smaller fragments are analysed, characterization might be impeded due to the unfavourable signal/background ratio. The advantage of the two dimensional electrophoretic technique is the development of compact spots that are distinguished from the background. It is also possible to separate the PCR fragments on a highly concentrated agarose gel instead of a PAGE if only the larger fragments are desired. Using the SSH technique of the present invention, only the unique artificially modified DNA fragments were isolated. Efficiency of the technique was demonstrated by the purity and specificity of the fragments, which were suitable for sequencing after the second electrophoretic separation, without clean-up procedure. Thus, a novel method for the identification of organisms that contain genetic modifications of unknown nature is presented. The application of the method is shown by using the mold Penicillium nalgiovense that was still alive after cheese production process was completed. The results obtained in accordance with the present invention lead to the expectation that this technique is capable of being use in variety of applications such as those described herein, in particular for monitoring food-containing organism, which are unlabeled as genetically modified.

In this embodiment, the method of the present invention can be performed with tester nucleic acid samples comprising or substantially consisting of cDNA or fragments thereof, or fragments of DNA of prokaryotic or viral origin as well as with, of course, genomic DNA fragments. The subject invention can also be used to identify and isolate common sequences between genomic DNA and any particular fragment of genomic DNA (or cDNA) cloned into plasmid, phage, viral, cosmid or YAC vectors. This approach can be applied to mapping chromosome aberrations (point mutations, deletions, insertions, transversions, etc.) in patients with certain hereditary diseases using cytogenetic chromosome mapping data and a set of recombinant vectors which contain DNA fragments covering the disease target region.

The methods of the present invention can further comprise the step of cloning and/or sequencing the identified nucleic acid fragments. Detailed descriptions of conventional methods, such as those employed in sequencing, the construction of vectors and plasmids, the insertion of genes encoding polypeptides or the corresponding antisense construct into such vectors and plasmids, the introduction of plasmids into host cells, and the expression and determination thereof of genes and gene products can be obtained from numerous publication, including Sambrook et al., (1989) Molecular Cloning: A Laboratory Manual, 2nd ed., Cold Spring Harbor Laboratory Press. Candidate nucleic acids or encoded polypeptides identified in such a manner can be validated by expressing them and observing the phenotype. A further embodiment of the screening method therefore comprises the overexpression or inhibition of expression of the identified candidate nucleic acid or encoded polypeptide in said cell, tissue or animal for their capability of inducing a responsive change in the phenotype of said cell, tissue or animal, wherein said phenotype is preferably related to a disorder. The responsive change in the phenotype of said cells can be observed by subjecting the cells, secreted factors thereof, or cell lysates thereof, to analyzing different parameters like cell proliferation, electrophysiological activity, DNA synthesis, out-growth of cells, cell migration, chemokinesis, chemotaxis, development of vessels, marker gene expression or activity, apoptosis and/or vitality, etc.

Hence, said identified, sequenced and/or cloned nucleic acid fragment preferably belongs to an infectious agent, a food contaminant, a gene responive to the presence, sensitivity or resistance to toxicants, health risk, or a gene involved in a disease. Most preferably, said diseases is cancer, hypertension, or diabetes.

In case the the nucleic acid sequence of the amplified DNA fragment does relate to an unknown gene, it is envisaged to clone the corresponding gene and to elucidate its function. Thus, in a further embodiment the method of the present invention further comprises using the identified, sequenced and/or cloned nucleic acid fragment as a probe for cloning the corresponding gene or full length cDNA. Methods which are well known to those skilled in the art can be used to obtain and probe genomic or cDNA libraries; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Furthermore, various DNA libraries are commercially available; see, e.g., Clontech.

Hence, in contrast to previous methods—which are aiming at and/or are restricted to the identification of a particular target sequence—the methods of the present invention described herein are capable of identifying and isolating a whole set of nucleic acid fragments and corresponding genes which are likely to be involved in the development of a certain phenotyp or symptom. This is an important improvement over methods described in the prior art, since most of complex phenotypes including disease symptoms and traits such as quantitative trait loci (QTL) are multigenic, i.e. two or more genes are involved.

The subject invention further concerns kits and compositions which contain, typically in separate packaging or compartments, the reagents such as driver nucleic acid samples, adapters and primers required for practicing the PCR suppression method of the subject invention. Such kits may optionally include the reagents required for performing PCR reactions, such as DNA polymerase, DNA polymerase cofactors, and deoxyribonucleotide-5′-triphosphates. Optionally, the kit may also include various polynucleotide molecules, DNA or RNA ligases, restriction endonucleases, reverse transcriptases, terminal transferases, various buffers and reagents, and antibodies that inhibit DNA polymerase activity. The kits may also include reagents necessary for performing positive and negative control reactions. The kit may also contain components for high through put (HTS) screening such microarrays, chips, multi-well plates and apparatus therefor. Optimal amounts of reagents to be used in a given reaction can be readily determined by the skilled artisan having the benefit of the current disclosure.

A variety of DNA polymerases can be used during PCR with the subject invention. Preferably, the polymerase is a thermostable DNA polymerase such as may be obtained from a variety of bacterial species, including Thermus aquaticus (Taq), Thermus thermophilus (Tth), Thermus filiformis, Thermus flavus, Thermococcus literalis, and Pyrococcus furiosus (Pfu). Many of these polymerases may be isolated from the bacterium itself or obtained commercially. Polymerases to be used with the subject invention can also be obtained from cells which express high levels of the cloned genes encoding the polymerase.

The subject invention can also be used with long distance (LD) PCR technology (Barnes, Proc. Natl. Acad. Sci. USA 91 (1994), 2216-2220; Cheng et al., Proc. Natl. Acad. Sci. USA 91 (1994), 5695-5699). LD PCR, which uses a combination of thermostable DNA polymerases, produces much longer PCR products with increased fidelity to the original template as compared to conventional PCR performed using Taq DNA polymerase alone.

The invented technique is capable of discovering nearly every differences in a genome compared to another. Therefore, the technique is most useful in identifying genes that, are etiologically related to disease. The results from such investigations will provide investigators with a wide range of possible genes for the elucidation of disease etiology, response to therapy and disease prevention.

Hence, the clinical applications of the methods of the present invention comprise, for example etiology and diagnosis, i.e. analysing the association of the presence or absence of certain genes with various diseases, association of the presence or absence of certain genes with different stagges of the same disease, association of the presence or absence of certain genes with the risk to develop various diseases under normal conditions (long term/short term), association of the presence or absence of certain genes with the risk to develop various diseases under exposure to physical, biological, and chemical toxicants (long term/short term), and association of the presence or absence of certain genes that are linked to a certain disease with a certain outcome of this disease after intervention

Furthermore, the described methods can be used for the prevention of disease (1.,2.,3. degree prevention), for example by analysing the association of the presence or absence of certain genes that are linked to a certain disease with a certain response to preventive measures, association of the presence or absence of certain genes that are linked to the development of a certain disease under normal conditions with a certain response to preventive measures, association of the presence or absence of certain genes that are linked to the development of a certain disease under exposure to physical, biological, and chemical toxicants with a certain response to preventive measures, association of the presence or absence of certain genes that are linked to a certain disease with a certain outcome of this disease that has a certain outcome without after intervention with a certain response to preventive measures, and association of the presence or absence of certain genes that are linked to a certain disease with a certain outcome of this disease that has a certain outcome after intervention with a certain response to preventive measures.

Moreover, the methods of the present invention can be used for improving drug response with pharmacogenomics. Adverse drug reactions, which in the USA are estimated to account for 100,000 hospitalizations annually, could be halved by the implementation of personalized medicine, for example by analysing a patient with a method of the present invention for the presence or absence of a gene involved in drug metabolism; see for review, e.g., Ferentz, Pharmacogenomics 3 (2002), 453-467. Thus, the method of the present invention can be applied advantageously throughout drug development to bring drugs successfully to market along with diagnostic tests that ensure their appropriate use.

In another embodiment the present invention relates to a method for diagnosing in a subject a phenotype, preferably disease or a predisposition to such a phenotype comprising:
(a) analyzing a sample of nucleic acids of a subject for example by means of a diagnostic chip, primer extension, single nucleotide polymorphisms, probe or sequencing comprising a nucleic acid molecule identified or cloned as described above, and
(b) comparing the result with that of a sample obtained from a subject displaying or known to develop the phenotype,
   wherein the presence or absence of said nucleic acid or the corresponding gene or cDNA is indicative for the phenotype or a corresponding predisposition. Similarly, a corresponding method may be used for analyzing a sample for the expression product of the mentioned nucleic acid molecule, for example by means of antibody.

In these embodiments, nucleic acid molecules, (poly)peptide, or antibodies are preferably detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or 125I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

In addition, the above-described nucleic acids, proteins, antibodies, etc. may be attached to a solid phase. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing nucleic acids, (poly)peptides, proteins, antibodies, etc. on solid phases include but are not limited to ionic, hydrophobic, covalent interactions and the like. The solid phase can retain one or more additional receptor(s) which has/have the ability to attract and immobilize the region as defined above. This receptor can comprise a charged substance that is oppositely charged with respect to the reagent itself or to a charged substance conjugated to the capture reagent or the receptor can be any specific binding partner which is immobilized upon (attached to) the solid phase and which is able to immobilize the reagent as defined above.

Commonly used detection assays can comprise radioisotopic or non-radioisotopic methods. These comprise, inter alia, RIA (Radioisotopic Assay) and IRMA (Immune Radioimmunometric Assay), EIA (Enzym Immuno Assay), ELISA (Enzyme Linked Immuno Assay), FIA (Fluorescent Immuno Assay), CLIA (Chemioluminescent Immune Assay), and electronic chip and array systems; see supra. Other detection methods that are used in the art are those that do not utilize tracer molecules. One prototype of these methods is the agglutination assay, based on the property of a given molecule to bridge at least two particles.

For diagnosis and quantification of (poly)peptides, polynucleotides, etc. in clinical and/or scientific specimens, a variety of immunological methods, as described above as well as molecular biological methods, like nucleic acid hybridization assays, PCR assays or DNA Enzyme Immunoassays (Mantero et al., Clinical Chemistry 37 (1991), 422-429) have been developed and are well known in the art. In this context, it should be noted that the nucleic acid molecules may also comprise PNAs, modified DNA analogs containing amide backbone linkages. Such PNAs are useful, inter alia, as probes for DNA/RNA hybridization.

The above-described compositions may be used for methods for detecting expression of a target gene by detecting the presence of mRNA which comprises, for example, obtaining mRNA from cells of a subject and contacting the mRNA so obtained with a probe/primer comprising a nucleic acid molecule capable of specifically hybridizing with the target gene under suitable hybridization conditions, and detecting the presence of mRNA hybridized to the probe/primer. Further diagnostic methods leading to the detection of nucleic acid molecules in a sample comprise, e.g., polymerase chain reaction (PCR), ligase chain reaction (LCR), Southern blotting in combination with nucleic acid hybridization, comparative genome hybridization (CGH) or representative difference analysis (RDA). These methods for assaying for the presence of nucleic acid molecules are known in the art and can be carried out without any undue experimentation.

Furthermore, the invention comprises methods of detecting the presence of a target gene product, i.e. a protein in a sample, for example, a cell sample, which comprises obtaining a cell sample from a subject, contacting said sample with one of the aforementioned antibodies under conditions permitting binding of the antibody to the protein, and detecting the presence of the antibody so bound, for example, using immuno assay techniques such as radioimmunoassay or enzymeimmunoassay. Furthermore, one skilled in the art may specifically detect and distinguish polypeptides which are functional target proteins from mutated forms which have lost or altered their activity by using an antibody which either specifically recognizes a (poly)peptide which has native activity but does not recognize an inactive form thereof or which specifically recognizes an inactive form but not the corresponding polypeptide having native activity.

Furthermore, the present invention relates to a method as described above wherein said sample is or is derived from hair, blood, serum, sputum, feces or another body fluid. The sample to be analyzed may be treated such as to extract, inter alia, nucleic acid molecules, (poly)peptides, or antibodies.

The present invention also relates to kit compositions containing specific reagents such as those described herein-before. Kits containing oligonucleotides, DNA or RNA, antibodies or protein may be prepared. Such kits are used to detect for example DNA which hybridizes to DNA of the target gene or to detect the presence of protein or peptide fragments in a sample. Such characterization is useful for a variety of purposes including but not limited to forensic analyses, diagnostic applications, and epidemiological studies in accordance with the above-described methods of the present invention. The recombinant target proteins, DNA molecules, RNA molecules and antibodies lend themselves to the formulation of kits suitable for the detection and typing of the target gene. Such a kit would typically comprise a compartmentalized carrier suitable to hold in close confinement at least one container. The carrier would further comprise reagents such as recombinant protein or antibodies suitable for detecting the expression or activity of the target gene or gene product. The carrier may also contain a means for detection such as labeled antigen or enzyme substrates or the like.

In summary, the present invention relates to the use of the described SSH assay techniques and their corresponding kits and components as well identified nucleic acid sequences for monitoring food, diagnosing polygenic phenotypes, forensic analysis, analysis of differences of closely related organisms, or any one of the above described applications.

These and other embodiments are disclosed and encompassed by the description and Examples of the present invention. Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

The above disclosure generally describes the present invention. A more complete under-standing can be obtained by reference to the following specific examples and figure which are provided herein for purposes of illustration only and are not intended to limit the scope of the invention. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature; see, for example, DNA Cloning, Volumes I and II (D. N. Glover ed. 1985) Oligonucleotide Synthesis (M. J. Gait ed.. 1984): Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986).

Detailed descriptions of conventional methods, such as those employed in the construction of vectors and plasmids, the insertion of genes encoding polypeptides into such vectors and plasmids, the introduction of plasmids into host cells, and the expression and determination thereof of genes and gene products can be obtained from numerous publication, including Sambrook et al., (1989) Molecular Cloning: A Laboratory Manual, 2nd ed., Cold Spring Harbor Laboratory Press.

The figures show:

FIG. 1: Enrichment of DNA fragments specifically for the GMO strains of P. nalgiovense. Gel electrophoresis in 2% Agarose. Stained with ethidium bromide. Lane 1: length standard lambda DNA digested with HindIII and Eco RI; Lane 2: Control P. nalgiovense BFE 19; Lane 3: Control P. nalgiovense BFE 20; Lane 4: Subtraction P. nalgiovense BFE 19 compared with P. nalgiovense BFE 66; Lane 5: Subtraction P. nalgiovense BFE 20 compared with P. nalgiovense BFE 66; Lane 6: Subtraction P. nalgiovense BFE 20 compared with P. nalgiovense BFE 328; Lane 7: Subtraction P. nalgiovense BFE 19 compared with P. nalgiovense BFE 328; Lane 8: Length standard pUC digest with Hpa II.

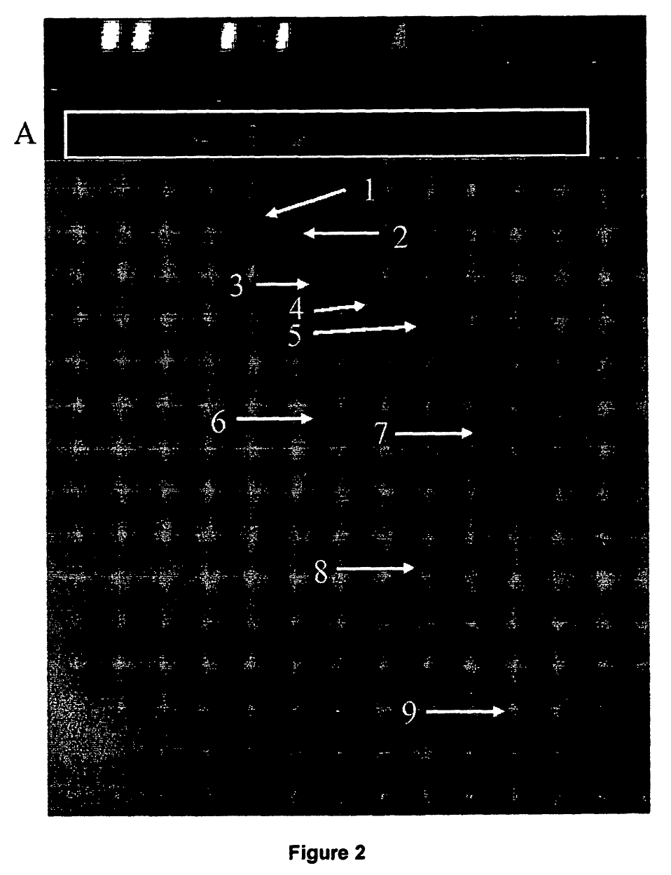

FIG. 2: Isolation of single DNA fragments by 2D-PAGE Gel electrophoresis in 6%+8% PA, DNA stained with silver. FIG. 2 A indicates the subtraction of P. nalgiovense BFE 19 compared with P. nalgiovense BFE 66 separated on a PAGE (6%). The 2D-pattem corresponds to the Agarose-Gel electrophoresis is framed in FIG. 1, lane 4. and indicates the region of interest electrophorized in PAGE in order to obtain a higher resolution of separated PCR fragments. The marked gene fragments show a high similarity (98-100%) to the following genes: listed in table 1.

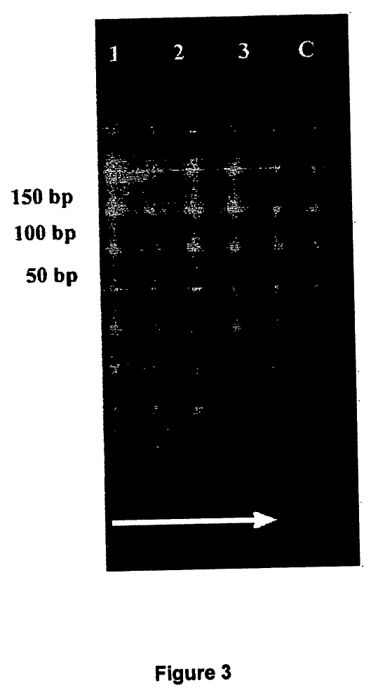

FIG. 3: Result of suppression PCR using different template concentrations after second hybridization step. 1) 10 μl; 2) 5 μl; 3) 1 μl template; c) unsubtracted control. The arrow indicates increasing template concentrations as described above.

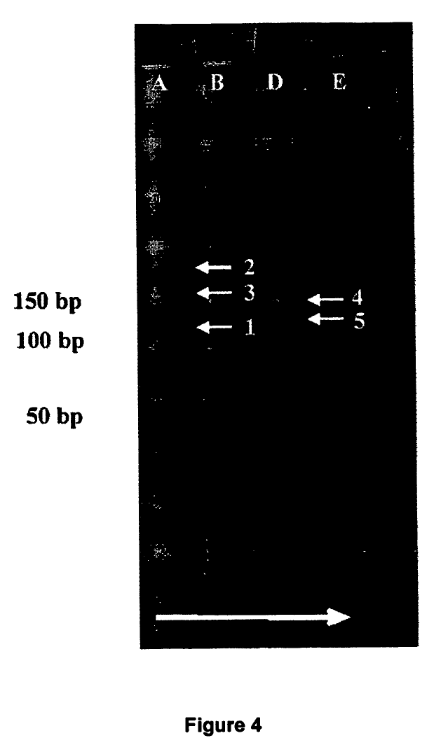

FIG. 4: Polyacrylamide gel electrophoresis for improvement of DNA fragments separation. The figure shows the migration of DNA fragments after enrichment PCR. Gel electrophoresis was carried out in a 6% polyacrylamide gel for 4 h. The arrow indicates increasing template concentrations as described above. A) 10 μl; B) 5 μl; C) 1μl template; D) unsubtracted control. 1-5 eluted Spots for adjacent purification using core sample PCR.

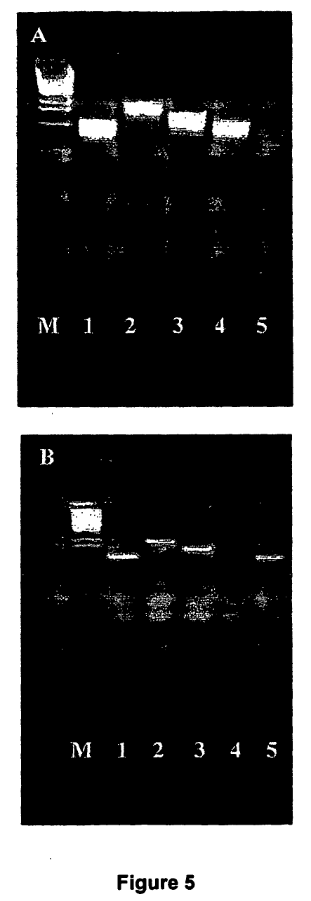

FIG. 5: Purification of DNA fragments using core sample PCR instead of bisbenzimide-PEG. A) PCR after eluting the fragments from the polyacrylamide gel; B) purified PCR fragments after adjacent core sample PCR. 1-5 samples are corresponding with the samples shown in FIG. 2; M length standard marker.

FIG. 6: Flowchart of experiment design for the analysis of two subjects differing phenotypically. The method of the present invention introduces the application of SSH inter alia on human nucleic acids, which includes DNA (genomic) as well as RNA (cDNA) and employs the analysis by the direct genomic comparison of two pools of DNA or RNA, respectively, which is attached to two different adaptors. DNA pool A and B are then hybridized separately to an excess of unligated driver DNA. Driver DNA is derived from a control sample, with an absent of certain genes or parts of genes caused by deletion or insertion mutations. An adjacent second hybridization steps is conducted by mixing, the two DNA pools with an excess of driver DNA. The duration of the second hybridization depends on the complexity of the DNA and may vary in different approaches. The enrichment of the unique DNA sequence/sequences is obtained by two PCR attempts.

### EXAMPLES

### Example 1

### Characterization of Minute Differences Between Genomes of Strains of Penicillium nalgiovense Using Subtractive Suppression Hybridization (SSH) Without Cloning

Penicillium nalviogensis (designated as BFE) was supplied by Bundesforschungsanstalt für Emährung, Karlsruhe; BFE. Strains BFE 19 and BFE 20 were used as tester organisms and were genetically modified by cloning the vector p3SR2 and pKW 100 (without further accessible data) which were incorporated into the genome at different locations. BFE 66 as well as BFE 328 were used as driver organisms. The molds were cultivated in malt medium and malt agar petridishes at 25° C. in the dark. Bacterial contamination was inhibited by adding Kanamycin (50 μg/ml) to the medium. DNA isolation was carried out according to Waver et al. (1995).

Subtractive Suppression Hybridization

The SSH procedure was performed with slight modifications as described (Diatchenko et al. (1996) using RSAI restriction endonuclease (Amersham Life Science). 5 μg of each genomic driver and tester DNA were digested with 15U RSAI in a volume of 50 μl. 1:5 diluted Tester DNA was divided into two pools and ligated to 10 μM adaptor 1 or 2R, respectively (Clontech) using 400 U T4 ligase. After ligation the DNAs were precipitated with ethanol, recovered and dissolved in 10 μl bidest.

First Hybridisation and Second Hybridisation

The first subtractive hybridization was carried out with 1.5 μl tester 1 or tester 2R and 1 μl hybridization buffer plus 1.5 μl driver DNA (excess 30 fold) (Clontech). The samples were covered with mineral oil and hybridized at 63° C. for 8 h with addition of fresh denatured driver DNA after 1 and 3 h. Enrichment of tester specific sequences was performed during a second hybridization by mixing the two primary hybridization samples together without denaturing and by adding 1 μl of fresh denatured driver DNA. The duration of hybridization was 60 h due to the complexity of the genome.

Polymerase Chain Reaction: Enrichment of Tester Specific Sequences

Prior to suppression PCR amplification the tester DNA was treated with DNA polymerase at 75° C. for 5 min. to add the complement of the defined adaptors. Suppression PCR amplification was carried out in volumes of 50 μl containing 20 mM Tris-HCl (pH 8.4), 50 mM KCl, 1.5 mM MgCl₂, 10 μmol of primer 1, 0.4mM of each dNTP and 2.5 units of Taq polymerase (Taq Polymerase, Gibco BRL) and 1 μl of subtractive hybridization DNA sample (diluted 1:40). Reactions were carried out in a Robocycler (Stratagene) with 30 cycles of 95° C. for 45 sec., 66° C. for 45 sec., and 72° C. for 90 sec. followed by a final extension step at 72° C. for 5 min. A secondary nested PCR amplification using of 1:10 diluted sample from the first PCR amplification reaction was carried out to further reduce background and enrich for tester specific DNA fragments. The nested PCR reaction followed the same condition as previously described above using the nested primers N1 and N2R with 11 cycles with an annealing temperature of 68° C.

Two-Dimensional Gel Electrophoresis

For the first electrophoretic step the PCR fragments were separated using a 6% PAGE. (Zhang et al. (1996) and Dotycz (1993)). After ethidium bromide treatment, the stained lane was excised with a scalpel and positioned perpendicularly to the running direction of a second gel for electrophoresis in the second dimension. The PAGE (8%) was equilibrated with 500 ml EDTA-buffer (25 mM, pH 5.9) for 1 h and stained with 100 ml EDTA-buffer containing bisbenzimide PEG (polyethylene glycol)-ether (150U) for 5 h at 55° C. in the dark prior to electrophoretic separation. The dye HA-Yellow was synthesized and is available from Hanse-Analytik (Fahrenheitstrasse 1, D-28359 Bremen, Germany Trademark HAY and HAR). Silver staining of the PAGE was carried out according to Allen et al. (1989). After staining with silver, characteristic spots were excised with a toothpick and further amplified with PCR according to the core amplifying method (core sample PCR). DNA was stabbed out of agarose from the centre of the bands with a sterile yellow pipette tip. The extracted DNA was used directly for a second PCR reaction with an initial denaturation step at 95° C. for 5 min. using the same nested primers as mentioned before. The amplified PCR fragments were electrophorized in an agarose gel stained with the dye PEGIII that retards the run of GC rich fragments. After elution from the gel, the fragments were sequenced and aligned via Internet with the sequence blast (Basic Local Alignment Search Tool) service of NCBI using the blast program blast 2.1 (Altschul et al. (1997)).

Results

FIG. 1 shows the gelelectrophoresis patterns after SSH of two genetically modified strains of P. nalgiovense (BFE 19 and BFE 20) using two different wild type strains as references (BFE 66 and BFE 328). The SSH lead to an enrichment of a multitude of DNA fragments in each of the four attempts. As shown in the figure, all SSH-PCR attempts revealed significant fragment patterns with high background which necessitated a two dimensional electrophoresis (Harms et al. (2000)). For further analysis of the SSH-PCR fragments, we used the PAGE (6%) to separate sequences because of its higher resolution of the PCR fragments. An example of the separation of single DNA fragments by 2D-PAGE is shown in FIG. 2. The arrows point at the DNA fragments, which are descended from genes of foreign species and the cloning vectors used for the transformation of the mold, respectively. The marked gene fragments show a high similarity (98-100%) to the following genes: listed in table 1.

TABLE 1
Sequences found in P. nalgiovense BFE 19 compared with
P. nalgiovense BFE 66

| Spot | Origin of sequences according to genebank | Source |
|---|---|---|
| 1 and 7 | Partial sequence of vector pJIR 751 | Cloning vector |
| | | p3SR2/pKW 100 |
| 2 | Aspergillus nidulans trp C | Cloning vector |
| | | p3SR2 |
| 3 | Partial sequence of vector pBACe | Cloning vector |
| | | p3SR2/pKW 100 |
| 4 | Partial sequence of bacterial gene | Cloning vector |
| | | p3SR2/pKW 100 |
| 5 | Partial sequence of bacterial gene | Cloning vector |
| | | p3SR2/pKW 100 |
| 6 | Partial sequence of pSOS | Cloning vector |
| | | p3SR2/pKW 100 |
| 8 and 9 | Aspergillus nidulans amd S | Cloning vector |
| | | p3SR2 |

A parallel agarose gel electrophoresis containing PEGIII to eliminate background produced the same resolution of separation. After PCR amplified fragments were aligned, nine hits were found for BFE 19 driven against BFE 66 and twelve for BFE 20 against BFE 66. The sequences had their origin in foreign species as well as in transformed vectors (FIG. 2). The fragments for instance were analysed as lacZ sequences of the vector pKW 100 as well as sequences from amdS and trpC from Aspergillus nidulans of the vector p3SR2.

### Example 2

### SSH (Subtractive Suppression Hybridization) Using Human Genomic DNA as Template

DNA isolation, restriction enzyme digestion of the DNA as well as the subtractive suppression hybridization was carried out as described in Example 1. First hybridization and second hybridization was performed with slightly modifications. The first subtractive hybridization was carried out with 1.5 μl tester 1 or tester 2R and 1 μl hybridization buffer plus 1.5 μl driver DNA (excess 30 fold) (Clontech). The samples were covered with mineral oil and hybridized at 63° C. for 16 h with addition of fresh denatured driver DNA after 2 and 5 h due to the high complexity of the human genome. Second hybridization was performed as described in Example 1.

Polymerase chain reaction: enrichment of tester specific sequences was carried out as described in Example 1 with slightly modifications. The template DNA was increased prior to suppression PCR attempt by diluting DNA samples 1:10 and applying 10, 5, 1 μl as template DNA, respectively; see FIG. 3.

Electrophoresis of PCR fragments: To improve the migration DNA fragments were separated on a 6% polyacrylamide gel after suppression PCR as shown in FIG. 4. Prominent spots were eluted by excision using a sterile scalpel. Subsequently, the gel pieces were boiled for 10 min. in a total volume of 100 μl sterile water. 5 μl of the sample were used for a following PCR amplification employing the same primer combination as for the enrichment PCR. The fragments were separated on a 1,5% agarose gel.

Purification of PCR fragments: Purification procedures were carried out as described in Example 1 with slightly modifications. After staining with ethidiumbromide, characteristic spots were excised with a toothpick and further amplified with PCR according to the core amplifying method (core sample PCR). In brief, DNA was stabbed out of agarose from the centre of the bands with a sterile yellow pipette tip diluted in 20 μl sterile water and the melted block containing the DNA fragments of interest was used directly for a second PCR reaction using the same nested primers as mentioned before (see FIG. 5A and 5B). By default of the chemical bisbenzimide-PEG the core sample PCR method is prioritized in order to purify the PCR fragments. It is undoubted that the application of the core sample PCR is on a par with the use of bisbenzimide-PEG.

### References

(1) Müller, W.; Hattesohl, I.; Schuetz, H.-J.; Meyer, G. (1981) Nucl.Acids Res. 9, 95-118.

(2) Harms C, Klarholz I and Hildebrandt A. (2000) Two-Dimensional Agarose Gel Electrophoresis as a Tool to Isolate Genus- and Species-Specific Repetitive DNA Sequences. Anal. Biochem. 284, 6-10.

(3) Wawer C, Ruggeberg H, Meyer G, Muyzer G. (1995) A simple and rapid electrophoresis method to detect sequence variation in PCR-amplified DNA fragments. Nucleic Acids Res. 11(23), 4928-9.

(4) Diatchenko, L., Lau, Y., Campbell, A. P., Chenchik, A., Moqadam, F., Huang, H., Lukyanov, S., Lukyanov, K., Gurskaya, N., Sverdlov, E. D., Siebert, P. D. (1996) Suppression subtractive hybridization: A method for generating differentially regulated or tissue-specific cDNA probes and libraries, Proc. Natl. Acad. Sci. USA 93, 6025-6030.

(5) Zhang, D., Yang, Y., Castlebury, L. A., Cerniglia, C. E. (1996) A method for the large scale isolation of high transformation efficiency fungal genomic DNA, FEMS Microbiology Letters 145,261-265.

(6) Allen, R. C., Graves, G., Budlowe, B. (1989) Polymerase Chain Reaction Amplification Products separated on rehydratable Polyacrylamide Gels and stained with Silver, BioTechniques 7, 736-744.

(7) Doktycz, M. J. (1993) Discontinuous Electrophoresis of DNA: Adjusting DNA Mobility by Trailing Ion Net Mobility, Analytical Biochemistry 213, 400406.

(8) Altschul S F, Madden T L, Schaffer A A, Zhang J, Zhang Z, Miller W, Lipman D J (1997) Gapped BLAST and PSI-BLAST: a new generation of protein database search programs. Nucleic Acids Res. 25(17), 3389-402.

(9) Diatchenko, L., Lukyanov, S., Lau, Y F. C. and Siebert, P. D. (1999). Suppression Subtractive hybridization: A Versatile Method for Identifying Differentially Expressed genes. Meth. Enzym. 303, 349-380.

(10) Lisitsyn, N., Lisitsyn, N. and Wigler, M. (1999). Cloning the Differences between Two Complex genomes. Science 259, 946 951.

(11) Hubank, M, Schatz D G. (1 999). DNA representational difference analysis: a sensitive and flexible method for identification of differentially expressed genes. Methods Enzymol.

1. A method for identifying and/or isolating a nucleic acid fragment or a corresponding gene which is unique for a certain cell, tissue or organism, comprising the steps of:
(a) dividing a tester nucleic acid sample into a first and a second nucleic acid sample, wherein said nucleic acids comprise or substantially consist of eukaryotic genomic DNA fragments;
(b) attaching a first PCR suppression adapter to each end of a DNA fragment in said first nucleic acid sample and attaching a second PCR suppression adapter to each end of a DNA fragment in said second nucleic acid sample;
(c) contacting each of said first and second nucleic acid samples separately with a driver nucleic acid sample;
(d) denaturing and reannealing said DNA fragments;
(e) combining said first and second nucleic acid samples to form a mixture of nucleic acids;
(f) contacting said nucleic acid mixture with a first nucleic acid primer comprising a nucleotide sequence that is complementary to a nucleotide sequence of said first adapter and contacting said nucleic acid mixture with said second nucleic acid comprising a nucleotide sequence that is complementary to a nucleotide sequence of said second adapter;
(g) adding to said mixture obtained after step (f) an effective amount of reagents necessary for performing a PCR; and
(h) cycling the mixture obtained after step (g) through at least one cycle of the denaturing, annealing and primer extension steps of PCR, wherein amplification of non-unique nucleic acid fragments is suppressed during PCR.

2. The method of claim 1, wherein the driver DNA is in excess to said tester DNAs.

3. The method of claim 1, wherein said driver nucleic acid sample comprises nucleic acid sequences that are complementary with at least one nucleic acid fragment in said first and second nucleic acid samples.

4. The method of claim 1, wherein step (c) further comprises filling in any single-stranded portions of said adapter after denaturing and reannealing of said nucleic acid fragments, wherein said adapter and said nucleic acid fragment comprise nucleic acid that is double-stranded.

5. The method of claim 1, wherein said nucleic acid fragments are less than 500 bp in length.

6. The method of claim 1, wherein said nucleic acid fragments result from restriction endonuclease digestion.

7. The method of claim 1, wherein the restriction endonuclease is RSAI.

8. The method of claim 1, wherein said nucleic acids of said tester or driver nucleic acid sample are immobilized or suspended on a chip or (micro)array.

9. The method of claim 1, wherein the driver nucleic acid sample comprises a pool of nucleic acids.

10. The method of claim 9, wherein said nucleic acids of said tester nucleic acid sample comprise or substantially consist of fragments of prokaryotic or viral DNA.

11. The method of claim 1, wherein said cells, tissue or organisms display different phenotypes.

12. The method of claim 1, wherein said samples are derived from the same or similar species.

13. The method of claim 1, wherein said samples are derived from the same or related subjects.

14. The method of claim 1, wherein said samples are derived from a vertebrate or a plant.

15. The method of claim 14, wherein said vertebrate is a mammal or a fish.

16. The method of claim 15, wherein said mammal is a human.

17. The method of claim 1, wherein said tester nucleic acid sample is derived from diseased tissue and said driver nucleic acid sample is derived from healthy tissue or vice versa.

18. The method of claim 1, wherein said unique nucleic acid fragment corresponds to a disease causing gene.

19. The method of claim 17, wherein said unique nucleic acid fragment or corresponding gene is present in the diseased tissue and absent in the healthy tissue or vice versa.

20. The method of claim 1, wherein said adapters or nucleic acid primers comprise a nucleotide sequence comprising restriction endonuclease recognition site.

21. The method of claim 1, further comprising subjecting the PCR fragments to 2D gel electrophoresis.

22. The method of claim 21, wherein said 2D gel electrophoresis comprises agarose and/or polyacrylamide gel electrophoresis.

23. The method of claim 20, wherein said nucleic acids of said tester nucleic acid sample comprise or substantially consist of cDNA or fragments thereof, or fragments of DNA of prokaryotic or viral origin.

24. The method of claim 1, further comprising cloning and/or sequencing the identified nucleic acid fragment.

25. The method of claim 1, wherein said identified, sequenced and/or cloned nucleic acid fragment belongs to an infectious agent, a food contaminant, a gene responsive to the presence, sensitivity or resistance to toxicants, health risk, or a gene involved in a disease.

26. The method of claim 25, wherein said diseases is cancer, hypertension, or diabetes.

27. The method of claim 1, further comprising using the identified, sequenced and/or cloned nucleic acid fragment as a probe for cloning the corresponding gene.

28. A method for diagnosing in a subject a phenotype, preferably a disease or a predisposition to such a phenotype comprising:
(a) analyzing a sample of a subject for the presence or absence of the nucleic acid fragment or the corresponding gene identified and/or cloned by the method of claim 1 or for the encoded gene product; optionally
(b) comparing the result with that of a sample obtained from a subject displaying or known to develop the phenotype;
wherein the presence or absence of said nucleic acid fragment, the corresponding gene or gene product is indicative for the phenotype or a corresponding predisposition.

29. A kit for use in a method of identifying and/or isolating a nucleic acid fragment or a corresponding gene which is unique for a certain cell, tissue or organism, said kit comprising a component selected from the group consisting of a driver nucleic acid sample, restriction endonucleases, adapters, polymerases, primers, PCR reagents, microarray, chip, multi-well plate, a nucleic acid primer or adapter, a nucleic acid fragment or gene obtained by the method of claim 1.

30. A method for monitoring food, diagnosing polygenic phenotypes, forensic analysis, or analysis of differences of closely related organisms, said method comprising identifying and/or isolating a nucleic acid fragment or a corresponding gene which is unique for a certain cell, tissue or organism in accordance with a method of claim 1.

31. A kit for use in a method of diagnosing in a subject a phenotype, preferably a disease or a predisposition to such a phenotype, said kit comprising a component selected from the group consisting of a driver nucleic acid sample, restriction endonucleases, adapters, polymerases, primers, PCR reagents, microarray, chip, multi-well plate, a nucleic acid primer or adapter, a nucleic acid fragment or gene obtained by the method of claim 1.
